(19) United States
(12) Patent Application Publication
(10) Pub. No.: US 2013/0210667 A1
(43) Pub. Date: Aug. 15, 2013

(54) Biomarkers for Predicting Kidney and Glomerular Pathologies

(71) Applicants: Brad H. Rovin, Columbus, OH (US); Haikady N. Nagaraja, Columbus, OH (US); Xiaolan Zhang, Columbus, OH (US)

(72) Inventors: Brad H. Rovin, Columbus, OH (US); Haikady N. Nagaraja, Columbus, OH (US); Xiaolan Zhang, Columbus, OH (US)

(73) Assignee: THE OHIO STATE UNIVERSITY, Columbus, OH (US)

(21) Appl. No.: 13/821,972

(22) PCT Filed: Sep. 9, 2011
(86) PCT No.: PCT/US11/50966

§ 371 Date: Apr. 30, 2013

Related U.S. Application Data
(60) Provisional application No. 61/381,594, filed on Sep. 10, 2010.
(60) Provisional application No. 61/508,747, filed on Jul. 18, 2011.

(51) Int. Cl. G01N 33/68; G06F 19/00

(52) U.S. Cl. CPC G01N 33/6893; G06F 19/36
USPC 506/9; 435/7.92; 506/18; 702/19

(57) ABSTRACT

Biomarkers for determining a kidney and glomerular pathologies and methods of using the same are described.

### RELATED APPLICATIONS

This application claims the priority to U.S. Provisional Application Nos. 61/381,594 filed Sep. 10, 2010, and Ser. No. 61/508,747, filed Jul. 18, 2011, the entire disclosures of which are expressly incorporated herein by reference.

### GOVERNMENT SUPPORT

The invention was made with government support from the National Institute of Diabetes and Digestive and Kidney Diseases (NIDDK) of the U.S. National Institutes of Health, Grant Numbers RO1 DK-074661 and DK-077331. The government may have certain rights in the invention.

### FIELD OF INVENTION

This application is directed to methods for predicting kidney and glomerular pathologies in patients. In particular this invention relates to methods for diagnosing and monitoring such pathologies as glomerular diseases and systemic lupus erythematosus (SLE) disease, including, for example, interstitial inflammation in lupus nephritis (LN) and interstitial fibrosis in lupus nephritis.

The field of this invention relates to computer systems and methods to identify classifiers using data obtained from urine and/or blood samples. The invention further encompasses the use of the classifiers and combinations of markers identified by the classifiers in a wide variety of applications including: diagnosis; prognosis; prediction of disease, stage of disease or disease risk; monitoring disease progression and/or regression; monitoring disease reoccurrence and identifying risk of disease reoccurrence; determining and/or predicting response to treatment and/or treatment outcomes; monitoring and/or predicting treatment compliance or non compliance and the like.

### BACKGROUND

The search for sensitive urinary biomarkers of kidney performance has attracted important efforts due to their potential value as clinical tools for detecting early signs of various diseases. In clinical nephrology, successful management of patients requires early detection and implementation of the most appropriate therapy. Available diagnostic methods do not allow detection of fibrosing and/or inflammatory changes at an early stage.

This is a continuing concern since inflammation is a major cause for the pathogenesis and progression of systemic autoimmune diseases such as systemic lupus erythematosus (SLE).

Lupus nephritis (LN) is one of the most serious manifestations of systemic lupus erythematosus (SLE) and usually arises within 5 years of diagnosis. Lupus nephritis is histologically evident in most patients with SLE, even those without clinical manifestations of renal disease. The symptoms of lupus nephritis are generally related to hypertension, proteinuria, and renal failure.

The principal goal of therapy in lupus nephritis is to normalize renal function or, at least, to prevent the progressive loss of renal function. Therapy differs depending on the pathologic lesion.

Systemic lupus erythematosus is characterized by periods of illness, called flares, and periods of wellness, or remission. While the warning signs of a flare in a patient can include one or more of increased fatigue, pain, rash, fever, abdominal discomfort, headache and dizziness, by the time the patient is experiencing these symptoms, there is already further damage being inflicted on the patient's body, and in particular, the kidney.

Systemic lupus erythematosus tends to be chronic and relapsing, often with symptom-free periods that can last for years. Since the course and episodes (i.e., flare-ups) of acute systemic lupus erythematosus is unpredictable, the prognosis varies widely. It has been found, however, that if the initial inflammation is controlled, the long-term prognosis is good. Therefore, early detection and treatment of kidney damage caused by systemic lupus erythematosus can reduce the incidence of severe kidney disease.

The therapy of lupus nephritis is based upon the pathology seen on kidney biopsy of patients with SLE who present with signs and symptoms of kidney involvement. The kidney biopsy is an invasive test, and is associated with a finite incidence of morbidity (bleeding, infection, pain) and even mortality. Because of these associated morbidities, the kidney biopsy cannot be used to follow patient response to therapy in a serial, prospective fashion. Furthermore, at the time of disease flare-ups, patients are often treated on the basis of the first biopsy and clinical signs and symptoms, as opposed to obtaining more tissue through another invasive procedure.

It would therefore be highly clinically relevant to develop non-invasive clinical tests that accurately reflect the histology of the kidney in patients with lupus nephritis, and could be applied prospectively to patients receiving therapy to assess response and make appropriate adjustments, and to patients who have a kidney flare-up, to initiate the appropriate intensity of therapy.

Such clinical tests that reflect kidney pathology without the need for a kidney biopsy would eliminate any morbidity and mortality from a biopsy.

Such clinical tests that reflect kidney pathology without the need for a kidney biopsy would potentially allow precise adjustment of treatments to the individual patient. Because the therapies for lupus nephritis are highly toxic, have severe side effects, including death, adjustment of therapy based on objective findings would be expected to reduce the morbidity and mortality of treatment significantly.

Such clinical tests that reflect kidney pathology without the need for a kidney biopsy would potentially allow precise adjustment of treatments to the individual patient. This would be expected to significantly improve the response to therapy, improve kidney function, and reduce the incidence of chronic kidney disease and end stage kidney disease requiring dialysis or transplantation in patients with lupus nephritis or other glomerular diseases.

Such clinical tests that reflect kidney pathology without the need for biopsy would potentially provide significant insights into the pathogenesis of lupus nephritis, which would open investigation to new treatments for this disease.

Therefore, the identification and validation of biomarkers that non-invasively accurately reflect kidney histology without a need for a kidney biopsy is a major unmet need for patients with lupus nephritis.

Whereas certain disease markers have been shown to predict outcome or response to therapy at a population level, they are not sufficiently sensitive or specific to provide adequate clinical utility in an individual patient. As a result, the first clinical presentation for more than half of the patients with SLE are the warning signs of a flare-up of the SLE.

It is also possible that the heterogeneity of the individual response to environmental risk factors induces a high variability in any SLE marker concentration. As a consequence, any biological information carried by a single inflammatory protein cannot be sufficient in providing a comprehensive representation of the inflammatory state, and may not be able to accurately identify the presence or extent of the disease. In view of such complexities, it is unlikely that an individual marker or approach will yield sufficient information to capture the true nature of the disease process.

Presently there are no clinically useful biomarkers that can be readily used to predict a kidney histology during a flare-up of kidney disease in systemic lupus erythematosus.

What are lacking are tools for predicting the likelihood that a particular patient will suffer from specific pathologic lesions of systemic lupus erythematosus nephritis

Also lacking are tools for profiling factors influencing sensitivity and resistance of patients to systemic lupus erythematosus therapeutic agents. Such tools would be predictive of treatment response of a patient to a particular drug, and would allow for increased predictability regarding chemosensitivity or chemoresistance of such patients to enable the design of optimal treatment regimens for individual patients. Such tools would likewise enable the identification of new drugs.

There is an urgent, yet still unmet, need for use in clinical medicine and biomedical research for improved non-invasive tools to identify kidney pathology in the active phase of the disease. The present invention addresses these and other shortcomings of the prior art.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### SUMMARY

In a first aspect, there is provided method for generating a result useful in diagnosing and non-invasively monitoring renal pathology using samples obtained from a mammalian subject. The method includes: obtaining a dataset associated with the samples, wherein the dataset comprises protein expression levels for at least two markers selected from the group consisting of: urine monocyte chemotactic protein-1 (uMCP-1), serum creatinine (Src), hepcidin (uHep), and proteinura expressed as a ratio of urine protein:creatine (uPCR); and inputting the dataset into an analytical process that uses the data to generate a result useful in diagnosing and monitoring the renal pathology.

In another aspect, there is provided a non-invasive, real-time method to assess renal pathology in a subject, comprising:

measuring, in samples from the subject, at least two markers selected from the group consisting of: urine monocyte chemotactic protein-1 (uMCP-1), serum creatinine (Src), hepcidin (uHep), and proteinura expressed as a ratio of urine protein:creatine (uPCR); where an elevated level thereof, as compared to a standard, is indicative of renal pathology.

In certain embodiments, the samples comprise urine and serum obtained from the subject at substantially the same time.

In certain embodiments, the kidney pathology comprises one or more of: glomerular diseases; systemic lupus erythematosus (SLE) disease; interstitial inflammation in lupus nephritis (LN); interstitial fibrosis in lupus nephritis (LN); renal-interstitial inflammation (INF); idiopathic immune-complex glomerulonephritis; pauci-immune necrotizing and crescentic glomerulonephritis; membranous glomerulopathy; diabetic glomerulosclerosis; IgA nephropathy; advanced chronic kidney disease; and glomerular basement membrane abnormalities.

In certain embodiments, the analytical process is a Linear Discriminant Analysis model. Further, in certain embodiments, the analytical process can include use of a predictive model. In certain embodiments, the analytical process comprises comparing the obtained dataset with a reference dataset.

In certain embodiments, the reference dataset comprises protein expression levels obtained from one or more healthy control subjects, or comprises protein expression levels obtained from one or more subjects diagnosed with renal-interstitial inflammation (INF).

In certain embodiments, the method further comprises obtaining a statistical measure of a similarity of the obtained dataset to the reference dataset.

In another aspect, there is provided herein a method for classifying a sample obtained from a mammalian subject, comprising: obtaining a dataset associated with the sample, wherein the dataset comprises expression levels for at least two markers selected from the group consisting of:

urine monocyte chemotactic protein-1 (uMCP-1), serum creatinine (Src), hepcidin (uHep), and proteinura expressed as a ratio of urine protein:creatine (uPCR); inputting the dataset into an analytical process that uses the data to classify the sample, wherein the classification is selected from the group consisting of a lupus nephritis classification, a healthy classification, a renal-interstitial inflammation classification, a no renal-interstitial inflammation classification, a medication exposure classification, a no medication exposure classification; and classifying the sample according to the output of the process.

In another aspect, there is provided herein a method for classifying a sample obtained from a mammalian subject, comprising:

obtaining a dataset associated with the sample, wherein the dataset comprises expression levels for at least two markers selected from the group consisting of: urine monocyte chemotactic protein-1 (uMCP-1), serum creatinine (Src), and hepcidin (uHep), proteinura expressed as a ratio of urine protein:creatine (uPCR);

inputting the data into a predictive model that uses the data to classify the sample, wherein the classification is selected from the group consisting of: a renal-interstitial inflammation classification, a no renal-interstitial inflammation classification, wherein the predictive model has at least one quality metric of at least 0.7 for classification; and,

classifying the sample according to the output of the predictive model.

In certain embodiments, the predictive model has a quality metric of at least 0.8 for classification. In certain embodiments, the predictive model has a quality metric of at least 0.9 for classification. In certain embodiments, the quality metric is selected from area-under-curve (AUC) and accuracy. In certain embodiments, the limits of the predictive model are adjusted to provide at least one of sensitivity or specificity of at least 0.7. In certain embodiments, the limits of the predictive model are adjusted to provide at least one of sensitivity or specificity of at least 0.9.

In certain embodiments, the method further comprises using the classification for diagnosis, staging, prognosis, kidney inflammation levels, assessing extent of progression, monitoring a therapeutic response, predicting a renal-interstitial inflammation (INF) episode, or distinguishing stable from unstable manifestations of renal-interstitial inflammation (INF).

In certain embodiments, the dataset further comprises quantitative data for one or more clinical indications.

In certain embodiments, the method comprises using a Linear Discriminant Analysis model or a Logistic Regression model, and the model comprises terms selected to provide a quality metric greater than 0.75.

In certain embodiments, the method further comprises obtaining a plurality of classifications for a plurality of samples obtained at a plurality of different times from the subject.

In another aspect, there is provided herein a method of analyzing a subject sample for one or more subject-derived markers selected to identify at least a beginning of a renal-interstitial inflammation (INF) and/or tubulointerstitial inflammation (TI) episode in patients with lupus nephritis (LN), comprising:

assaying the sample for the presence or amount of subject-derived markers related to a INT or TI episode, wherein at least two markers are selected from the group consisting of: urine monocyte chemotactic protein-1 (uMCP-1), serum creatinine (Src), hepcidin (uHep), and proteinura expressed as a ratio of urine protein:creatine (uPCR); and

characterizing the subject's risk of having, or at risk for having, the INF and/or TI episode based upon the presence or amount of the markers.

In another aspect, there is provided herein a method for assigning a therapy regimen and/or assigning a prognosis to a subject diagnosed with or suspected of suffering from an interstitial inflammation episode, comprising:

performing an assay on a sample obtained from the subject, wherein the assay provides one or more detectable signals related to the presence or amount of one or more subject-derived markers independently selected from the group consisting of markers related to kidney flare episodes, or markers related to the subject-derived markers;

wherein at least two markers are selected from the group consisting of:

urine monocyte chemotactic protein-1 (uMCP-1), serum creatinine (Src), hepcidin (uHep), and proteinura expressed as a ratio of urine protein:creatine (uPCR); and

correlating the signal(s) obtained from the assay method to ruling in or out a therapy regimen for the subject and/or assigning a prognosis to the subject. In a particular embodiment, wherein the markers consist of: urine monocyte chemotactic protein-1 (uMCP-1) and serum creatinine (Src).

In another aspect, there is provided herein a method for assigning a therapy regimen and/or assigning a prognosis to a subject diagnosed with or suspected of suffering from interstitial fibrosis, comprising:

performing an assay on a sample obtained from the subject, wherein the assay provides one or more detectable signals related to the presence or amount of one or more subject-derived markers independently selected from the group consisting of markers related to kidney flare episodes, or markers related to the subject-derived markers;

wherein at least two markers are selected from the group consisting of:

urine monocyte chemotactic protein-1 (uMCP-1), serum creatinine (Src), hepcidin (uHep), and proteinura expressed as a ratio of urine protein:creatine (uPCR); and

correlating the signal(s) obtained from the assay method to ruling in or out a therapy regimen for the subject and/or assigning a prognosis to the subject. In a particular embodiment, the markers consist of: hepcidin (uHep) and proteinura expressed as a ratio of urine protein:creatine (uPCR).

In certain embodiments, the method rules in or out an assignment of the subject to early goal-directed therapy. In certain embodiments, the method rules in or out one or more treatments for inclusion in a therapy regimen comprising administration of immunosuppressive therapy.

Further, in certain embodiments, the correlating step comprises comparing one or more subject-derived marker concentrations to a predetermined threshold level for a particular marker of interest. In certain embodiments, the correlating step comprises: determining the concentration of the subject-derived markers,

calculating a single response value based on the concentration of the subject-derived markers, and comparing the response value to one or more predetermined threshold levels for the response value.

In certain embodiments, the correlating step comprises:

comparing the subject-derived marker concentrations to a predetermined threshold level for a particular marker of interest and determining the concentration of the subject-derived markers,

calculating a single response value based on the concentration of each of the subject-derived markers, and

comparing the response value to a predetermined threshold level for the panel response value.

In certain embodiments, the sample is from a human.

In certain embodiments, the assay method comprises an immunoassay.

In another particular aspect, there is provided herein a method for diagnosing a disease condition characterized by altered levels of at least two markers selected from the group consisting of: urine monocyte chemotactic protein-1 (uMCP-1), serum creatinine (Src), hepcidin (uHep), and proteinura expressed as a ratio of urine protein:creatine (uPCR). The method includes:

contacting a sample from a subject with an antibody or fragment thereof that specifically binds to one or more binding sites on the marker, and

quantifying the marker levels in the sample; wherein the altered levels of the markers is indicative of the disease condition. In certain embodiments, the antibody specifically binds an epitope contained within the marker.

In another aspect, there is provided herein a kit for detecting a disease condition characterized by non-physiological levels of one or more markers selected from urine monocyte chemotactic protein-1 (uMCP-1) and serum creatinine (Src), comprising, an anti-marker antibody or fragment thereof that specifically binds to one or more mid-portion or carboxy terminal epitopes of the marker, and a reagent that binds directly or indirectly to the antibody or fragment thereof. In certain embodiments, the anti-marker antibody or fragment thereof is immobilized on a support.

The system includes a non-invasive and easily accessible method for monitoring structural kidney changes or inflammation in lieu of an invasive kidney biopsy to predict kidney pathology in patients suffering from lupus and thereby directing therapy appropriately.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B: Urine biomarker levels as a function of degree of interstitial inflammation. Urine MCP-1 (FIG. 1A), urine Hepcidin (FIG. 1B), and urine LFABP (FIG. 1B) were measured in patients with lupus nephritis and segregated into groups with no-mild interstitial inflammation and moderate-severe interstitial inflammation. The graphs show mean+SEM urine cytokine levels. *P<0.0001 for uMCP-1, *P<0.003 for uHepcidin, *P<0.002 for uLFABP, moderate-severe inflammation vs. none-mild inflammation.

FIGS. 2A-2C: Individual urine biomarker levels as a function of degree of interstitial inflammation. Each patient's urine MCP-1 (FIG. 32A), urine Hepcidin (FIG. 2B), and urine LFABP (FIG. 2C) are shown after segregation into groups with no-mild interstitial inflammation and moderate-severe interstitial inflammation to illustrate the degree of overlap between the groups.

FIG. 3: Receiver-operating characteristic curve for a composite biomarker of renal interstitial inflammation. This ROC curve is based on Equation (1) [Eq(1) Y1=0.992*ln(uMCP1)+2.213*ln(Scr)], which combines uMCP-1, and Scr to differentiate biopsies with no-mild interstitial inflammation from moderate to severe interstitial inflammation. The area under the curve is 0.92.

FIG. 4: Receiver-operating characteristic curve for a composite biomarker of renal interstitial fibrosis. This ROC curve is based on Equation (2) [Eq.2 Y2=4.177*ln(uPCR)-1.425*ln(uHEP)], which combines uHep, and uPCR to differentiate biopsies with no-mild interstitial fibrosis from moderate to severe interstitial fibrosis. The area under the curve is 0.74.

### DETAILED DESCRIPTION OF THE INVENTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

All publications, patent applications, patents, and other references mentioned herein are incorporated by reference in their entirety. The disclosure of all patents, patent applications (and any patents that issue thereon, as well as any corresponding published foreign patent applications), GenBank and other accession numbers and associated data, and publications mentioned throughout this description are hereby incorporated by reference herein. It is expressly not admitted, however, that any of the documents incorporated by reference herein teach or disclose the present invention.

The present invention may be understood more readily by reference to the following detailed description of the embodiments of the invention and the Examples included herein. However, before the present methods, compounds and compositions are disclosed and described, it is to be understood that this invention is not limited to specific methods, specific cell types, specific conditions, etc., as such may, of course, vary, and the numerous modifications and variations therein will be apparent to those skilled in the art. It is also to be understood that the terminology used herein is for the purpose of describing specific embodiments only and is not intended to be limiting.

The present invention is based, at least in part, on the inventors' discovery of biomarkers of kidney pathology by focusing on biomarkers that reflect distinct pathologic lesions that are potentially important treatment targets to prevent chronic kidney disease.

This approach is more specific and flexible than correlating biomarkers to the International Society of Nephrology/Renal Pathology Society (ISN/RPS) classification classes of LN, and more useful, in that there can be broad variations in the histology within the same LN class, and combinations of classes are not infrequent. Additionally, the ISN/RPS schema does not address all the compartments of the kidney equally, but mainly considers glomerular changes.

### DEFINITIONS

Unless otherwise defined, all technical and scientific terms used herein have the same meaning as commonly understood by one of ordinary skill in the art to that this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

“Ameliorating” refers to any therapeutically beneficial result in the treatment of a disease state, e.g., an SLE disease state, including prophylaxis, lessening in the severity or progression, remission, or cure thereof.

“Correlating,” is used in reference to the use of diagnostic and markers, refers to comparing the presence or amount of the marker(s) in a patient to its presence or amount in persons known to suffer from, or known to be at risk of, a given condition; or in persons known to be free of a given condition. A marker level in a patient sample can be compared to a level known to be associated with a specific diagnosis. That is, the skilled artisan can use the marker level to determine whether the patient suffers from a specific type diagnosis, and respond accordingly. Alternatively, the sample's marker level can be compared to a marker level known to be associated with a good outcome (e.g., the absence and/or remission of disease, etc.). In certain embodiments, a profile of marker levels are correlated to a global probability or a particular outcome using ROC curves.

“Determining the diagnosis” refers to methods by which the skilled artisan can determine the presence or absence of a particular disease or condition (e.g., INF) in a patient. The term “diagnosis” does not refer to the ability to determine the presence or absence of a particular disease with 100% accuracy, or even that a given course or outcome is more likely to occur than not. Instead, the skilled artisan will understand that the term “diagnosis” refers to an increased probability that a certain disease is present in the subject. In certain embodiments, a diagnosis indicates about a 5% increased chance that a disease is present, about a 10% chance, about a 15% chance, about a 20% chance, about a 25% chance, about a 30% chance, about a 40% chance, about a 50% chance, about a 60% chance, about a 75% chance, about a 90% chance, about a 95% chance, about a 97% chance, and about 99% chance.

“Diagnosis” refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition. Also, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing interstitial kidney damage in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. Preferred prognostic markers can predict the onset of a flare-up in a patient, or the chance of future flare-up. Similarly, a prognostic risk signals a probability (“a likelihood”) that a given course or outcome will occur. A level or a change in level of a prognostic indicator, which in turn is associated with an increased probability of morbidity (e.g., worsening renal function, future SLE, or death) may be referred to as being “indicative of an increased likelihood” of an adverse outcome in a patient.

“Mammal” as used herein includes both humans and non-humans and include but is not limited to humans, non-human primates, bovines, canines, equines, felines, murines, and porcines.

“Monitoring” as used herein refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication. In particular, the term “monitoring” can refer to SLE staging, SLE prognosis, inflammation levels, assessing extent of SLE progression, monitoring a therapeutic response, predicting an SLE flare, or distinguishing stable from unstable manifestations of the SLE disease.

“Purified,” when used herein in the context of nucleic acids or proteins, denotes that a nucleic acid or protein gives rise to essentially one band in an electrophoretic gel. Particularly, it means that the nucleic acid or protein is at least 50, 55, 60, 65, 70, 75, 80, 85, 90, 91, 92, 93, 94, 95, 96, 97, 98, 99 or 100% pure with respect to the presence of any other nucleic acid or protein species.

“Quantitative data” as used herein refers to data associated with any dataset components (e.g., protein markers, clinical indicia, metabolic measures, or genetic assays) that can be assigned a numerical value. Quantitative data can be a measure of the DNA, RNA, or protein level of a marker and expressed in units of measurement such as molar concentration, concentration by weight, etc. For example, if the marker is a protein, quantitative data for that marker can be protein expression levels measured using methods known to those skilled in the art and expressed in mM or mg/dL concentration units.

“Subject” refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care. This includes persons with no defined illness who are being investigated for signs of pathology.

“Substrate” refers to a support, such as a rigid or semi-rigid support, to which nucleic acid molecules or proteins are applied or bound, and includes membranes, filters, chips, slides, wafers, fibers, magnetic or nonmagnetic beads, gels, capillaries or other tubing, plates, polymers, and microparticles, and other types of supports, which may have a variety of surface forms including wells, trenches, pins, channels and pores.

“Sufficient amount” means an amount sufficient to produce a desired effect, e.g., an amount sufficient to alter a protein expression profile.

“Test sample” refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. A sample may comprise a bodily fluid; a cell; an extract from a cell, chromosome, organelle, or membrane isolated from a cell; genomic DNA, RNA, or cDNA in solution or bound to a substrate; or a biological tissue or biopsy thereof. A sample may be obtained from any bodily fluid (blood, serum, plasma, urine, cerebrospinal fluid saliva, phlegm, gastric juices, sputum, pleural effusions, etc.), cultured cells, biopsies, or other tissue preparations. In addition, one of skill in the art would realize that some test samples would be more readily analyzed following a fractionation or purification procedure, for example, separation of whole blood into serum or plasma components.

“Therapeutically effective amount” is an amount that is effective to ameliorate a symptom of a disease. A therapeutically effective amount can be a “prophylactically effective amount” as prophylaxis can be considered therapy.

The methods described herein should not be interpreted to mean that the kidney injury marker assay result(s) is/are used in isolation in the methods described herein. Rather, additional variables or other clinical indicia may be included in the methods described herein. For example, a risk stratification, diagnostic, classification, monitoring, etc. method may combine the assay result(s) with one or more variables measured for the subject selected from the group consisting of demographic information (e.g., weight, sex, age, race), medical history (e.g., family history, type of surgery, pre-existing diseases, clinical variables (e.g., blood pressure, temperature, respiration rate), risk scores, and the like.

The present invention may be understood more readily by reference to the following detailed description of the embodiments of the invention and the Examples included herein. However, before the present methods, compounds and compositions are disclosed and described, it is to be understood that this invention is not limited to specific methods, specific cell types, or specific conditions, etc., as such may, of course, vary, and the numerous modifications and variations therein will be apparent to those skilled in the art. It is also to be understood that the terminology used herein is for the purpose of describing specific embodiments only and is not intended to be limiting.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The disclosure of all patents, patent applications (and any patents that issue thereon, as well as any corresponding published foreign patent applications), and associated data, and publications mentioned throughout this description are hereby incorporated by reference herein. It is expressly not admitted, however, that any of the documents incorporated by reference herein teach or disclose the present invention.

### EXAMPLES

The disclosure provides methods, compositions and kit for generating a result useful in diagnosing and monitoring kidney pathologies using one or more samples obtained from a mammalian subject.

In addition to the specific biomarker sequences identified in this application by name, accession number, or sequence, the invention also contemplates use of biomarker variants that are at least 90% or at least 95% or at least 97% identical to the exemplified sequences and that are now known or later discovered and that have utility for the methods of the invention. These variants may represent polymorphisms, splice variants, mutations, and the like.

After the dataset has been obtained it is preferably input into an analytical process that uses the quantitative data to generate a result useful in diagnosing and monitoring a disease state.

One preferred forms of the analytical process is a Linear Discriminant Analysis model. The analytical process may also use a predictive model or may involve comparing the obtained dataset with a reference dataset. In certain aspects, the reference dataset may be data obtained from one or more healthy control subjects or from one or more subjects. Comparing the reference dataset to the obtained dataset may include obtaining a statistical measure of a similarity of the obtained dataset to the reference dataset, which may be a comparison of the parameters of the obtained dataset to corresponding parameters from the reference dataset.

In certain aspects, the classes may be an active disease state classification, an incipient flare state, a healthy classification, a medication exposure classification, and a no medication exposure classification. In a specific embodiment, the class can be identification of renal-interstitial inflammation (INF) in patients with lupus nephritis (LN).

Also, a preferred analytical processes will provide a quality metric of at least 0.7, at least 0.75, at least 0.8, at least 0.85, or at least 0.9, where preferred quality metrics are area under the curve (AUC) and accuracy. Additionally, preferred analytical processes will provide at least one of sensitivity or specificity of at least 0.65, at least 0.7, at least 0.75 or at least 0.85.

The methods disclosed herein may be used, for example, for classification for diagnosis, staging, prognosis, inflammation levels, assessing extent of disease progression, monitoring a therapeutic response, or distinguishing stable from unstable manifestations of the SLE. In a specific embodiment, the class can be identification of renal-interstitial inflammation (INF) in patients with lupus nephritis (LN). In addition to the other markers disclosed herein, the markers may be selected from one or more clinical indicia.

The detection of levels of the markers identified herein, which are specifically produced as a result of the SLE disease process, can classify patients as belonging to SLE conditions, including active, or flare state, of the SLE disease, treatment with medication, no treatment, renal-interstitial inflammation (INF), and the like. Such classification can also be used in prediction of SLE flare events and response to therapeutics; and are useful to predict and assess complications of the SLE disease.

In one embodiment of the invention, the expression profile of a panel of proteins is evaluated for conditions indicative of various stages of SLE and clinical sequelae thereof. Such a panel provides a level of discrimination not found with individual markers. In one embodiment, the expression profile is determined by measurements of protein concentrations or amounts.

Methods of analysis may include, without limitation, utilizing a dataset to generate a predictive model, and inputting test sample data into such a model in order to classify the sample according to an SLE classification, and classifying the sample according to the output of the process. In some embodiments, such a predictive model is used in classifying a sample obtained from a mammalian subject by obtaining a dataset associated with a sample.

In one embodiment, the invention utilizes quantitative data, such as protein expression levels, from one or more sets of markers described herein. In some embodiments a predictive model provides for a level of accuracy in classification; i.e. the model satisfies a desired quality threshold. A quality threshold of interest may provide for an accuracy or area-under-the-curve (AUC) of a given threshold, and either or both of these terms (AUC; accuracy) may be referred to herein as a quality metric. A predictive model may provide a quality metric, e.g., accuracy of classification or AUC, of at least about 0.7, at least about 0.8, at least about 0.9, or higher. Within such a model, parameters may be appropriately selected so as to provide for a desired balance of sensitivity and selectivity.

In other embodiments, analysis of the markers is used in a method of screening biologically active agents for efficacy in the treatment of SLE. In such methods, cells associated with SLE are contacted in culture or in vivo with a candidate agent, and the effect on expression of one or more of the markers, e.g. a panel of markers, is determined. In another embodiment, analysis of differential expression of the markers is used in a method of following therapeutic regimens in patients. In a single time point or a time course, measurements of expression of one or more of the markers, e.g. a panel of markers, is determined when a patient has been exposed to a therapy, which may include a drug, combination of drugs, non-pharmacologic intervention, and the like.

In another method, relative quantitative measures of the SLE-associated proteins identified herein are used to diagnose or monitor an SLE disease in an individual. This panel of markers identified herein can further include other clinical indicia; additional protein expression profiles; metabolic measures, genetic information, and the like.

In another embodiment, the invention includes methods for classifying a sample obtained from a mammalian subject by obtaining a dataset associated with a sample, wherein the dataset comprises protein expression levels for markers, inputting the data into an analytical process that uses the data to classify the sample (where the classification is selected from the classification groups described herein), and classifying the sample according to the output of the process.

In another aspect, there is provided a method which comprises collecting a plurality of samples from a subject over time, and thereafter determining any change in levels of expression of one or more of the markers.

The method further comprises comparing the expression profiles of a baseline level with one or more subsequent levels to determine whether there is an altered expression of any of the expression levels.

In another aspect, there is provided herein methods for determining the impending remission of systemic lupus erythematosus and/or an impending kidney nephritis flare-up episode in a subject. Disease course refers to changes in disease status over time, including disease progression (worsening) and disease regression (improvement). Over time, the amounts or relative amounts (e.g., the pattern) of the markers changes. Therefore, the trend of these markers, either increased or decreased over time toward diseased or non-diseased indicates the course of the disease. The method includes the measurement of the markers in a subject for at least two different time points, e.g., a first time and a second time, and comparing the change in amounts, if any. The course of disease is determined based on these comparisons.

In another aspect, there is provided a method of analyzing a subject sample for one or more subject-derived markers selected to identify subjects suffering from a kidney nephritis flare-up episode, comprising: assaying the sample for the presence or amount of one or more subject-derived markers related to systemic lupus erythematosus, and characterizing the subject's risk of suffering a kidney nephritis flare episode based upon the presence or amount of the markers, wherein the amount of each of the markers is not compared to a predetermined threshold amount.

In a particular aspect, the method for assigning a therapy regimen and/or assigning a prognosis to a subject diagnosed with or suspected of suffering from systemic lupus erythematosus, comprising: performing an assay method on a sample obtained from the subject, wherein the assay method provides one or more detectable signals related to the presence or amount of one or more subject-derived markers independently selected from the group consisting of markers related to kidney flare episodes, or markers related to the subject-derived markers; and correlating the signal(s) obtained from the assay method to ruling in or out a therapy regimen for the subject and/or assigning a prognosis to the subject.

In another particular aspect, there is provided a method which can be useful as a tool to rule in or out an assignment of the subject to an early goal-directed therapy.

The method can include measuring a plurality of markers in the biological sample.

In certain embodiments, the one or more markers can be measured by mass spectrometry, such as SELDI-TOF. Also, the correlating can be performed by executing a software classification algorithm.

In another particular aspect, there is provided a method for reporting the status to the subject, and/or recording the status on a tangible medium.

In another particular aspect, there is provided a method for managing subject treatment based on such classification. Also, the method can further comprise measuring the at least one marker after subject management and correlating the measurement with systemic lupus erythematosus disease progression.

In a particular embodiment, the method includes (a) measuring, at a first time, one or more markers in a biological sample from the subject; (b) measuring, at a second time, at least one marker in a biological sample from the subject; and (c) comparing the first measurement and the second measurement; wherein the comparative measurements determine the course of the systemic lupus erythematosus.

The invention may be better understood by reference to the following examples, which serve to illustrate but not to limit the present invention.

### Example 1

The inventors herein identified urine biomarkers that can be used as surrogates for specific pathologic kidney lesions.

Thus, by mathematically combining these urine biomarkers, the inventors have discovered a combinatorial biomarker that provides very good sensitivity and specificity for determining whether a subject has a little or a lot of interstitial inflammation in their kidneys, without need to do a biopsy. This combinatorial biomarker panel is clinically important because biopsies are invasive and cannot be repeated frequently to assess what is going on in the kidneys, and interstitial damage, which is generally preceded by interstitial inflammation, is the most important risk factor for developing chronic kidney disease and end stage kidney disease.

While linear discriminant analysis may be used to construct an analytical process and/or increase the speed and efficiency of the application of the analytical process and to avoid investigator bias, one of ordinary skill in the art will realize that computer-based algorithms are not required to carry out the methods of the present invention.

Results

Biopsy Cohorts

Kidney biopsies were done for the clinical diagnosis of glomerular disease in 61 patients. All biopsies showed immune-complex glomerulonephritis consistent with LN. The entire biopsy population is described in Table 1.

TABLE 1
Cohort Characteristics

| | |
|---|---|
| Male/Female | 11/50 |
| Age (range) | 30 (17-51) |
| African American (%) | 26 (43%) |
| Caucasian (%) | 28 (46%) |
| Other Race/Ethnicity (Asian, Hispanic, Middle | 7 (11%) |
| Eastern) | |
| SLE Class at Biopsy | |
| 2 | 6 |
| 3 | 9 |
| 4 | 27 |
| 5 | 6 |
| 3 + 5 | 6 |
| 4 + 5 | 10 |
| Median Urine Protein to Creatinine Ratio (range) | 2.3 (0.24-16.5) |
| Median Serum Creatinine, mg/dl (range) | 0.93 (0.38-5.98) |
| Median Prednisone Dose at Urine Collection, mg/d | 17.5 (0-60) |
| (range) | |
| Median MMF Dose at Urine Collection, g/d (range) | 0 (0-3000) |
| Median AZA Dose at Urine Collection, mg/d (range) | 0 (0-150) |
| Pulse Corticosteroids Given Before Urine Collection | 12 (19%) |
| (% patients) | |

The patients with moderate-severe interstitial inflammation were directly compared to the patients with no or mild interstitial inflammation (Table 2).

TABLE 2
Comparison of Patients with Different Levels of
Interstitial Inflammation

| Demographic/Clinical | No-Mild | Moderate-Severe |
|---|---|---|
| Feature | Inflammation | Inflammation |
| Caucasian (%) | 25 (53) | 4 (24) |
| African American (%) | 16 (34) | 11 (64)¹ |
| Other (%) | 6 (13) | 2 (12)1 |
| Age | 30 ± 1.1 | 32 ± 2.1 |
| Mean SCr² (mg/dl) | 1.09 ± 0.10 | 2.61 ± 0.37 |
| Mean uPCR³ | 2.80 ± 0.42 | 5.33 ± 1.23⁴ |
| Class II (%) | 6 (13%) | 0 |
| Class III, III + V (%) | 11 (23%) | 4 (23.5%) |
| Class IV, IV + V (%) | 25 (53%) | 12 (70.5%) |
| Class V (%) | 5 (11%) | 1 (6%) |

¹African American plus other races/ethnicities were significantly over-represented in the moderate-severe interstitial inflammation group (P = 0.048, Fisher's exact test)
²Serum creatinine ± SEM
³Urine protein creatinine ratio
⁴P < 0.0001 vs no-mild inflammation (Mann-Whitney test)

African Americans and other non-Caucasians were over-represented in the moderate-severe inflammation group. Patients with moderate-severe inflammation had significantly more proteinuria at biopsy than patients with none-mild interstitial inflammation. Serum creatinine was numerically higher at the time of biopsy in patients with moderate-severe inflammation, but this did not reach significance. Similarly, there was a higher, but non-significant proportion of patients with Class IV or IV+V LN in the moderate-severe group.

Candidate Biomarkers of Interstitial Inflammation

Three candidate biomarkers were selected and examined for correlation to interstitial inflammation. These biomarkers were urine monocyte chemoattractant protein-1 (uMCP-1), urine Hepcidin (uHepcidin), and urine liver-type fatty acid binding protein (uLFABP).

uMCP-1 is a biomarker of active LN, and MCP-1 is made by infiltrating interstitial leukocytes in a number of glomerular diseases.

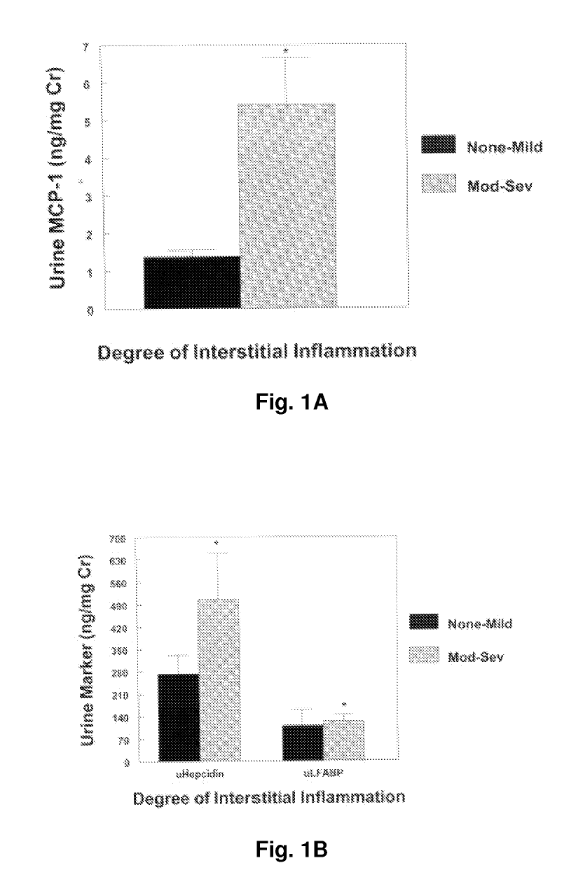

As shown in FIG. 1A, uMCP-1 was significantly greater in patients with moderate-severe interstitial inflammation than patients with no or mild interstitial inflammation. When used to classify the severity of interstitial inflammation in this test set of biopsies, uMCP-1 misclassified 10 of 64 biopsies (Table 3).

TABLE 3
Performance Characteristics of Biomarkers of Interstitial
Inflammation for all the Biopsies¹

| | Threshold | Misclassifications | Sens | Spec | PPV | NPV | AUC Under |
|---|---|---|---|---|---|---|---|
| Biomarker | Value² | (%) | (%) | (%) | (%) | (%) | ROC |
| uMCP-1 | 2.2 | 10/64 (16) | 83 | 85 | 67 | 93 | 0.87 |
| uLFABP | 118 | 14/64 (22) | 65 | 83 | 58 | 87 | 0.75 |
| uHepcidin | 136.5 | 22/64 (34) | 83 | 60 | 42 | 90 | 0.70 |
| uPCR | 3.7 | 18/61 (30) | 56 | 76 | 45 | 83 | 0.65 |
| Scr | 1.43 | 13/64 (20) | 83 | 79 | 58 | 93 | 0.86 |
| Eq (1) Y1 | 1 | 9/64 (14) | 100 | 81 | 67 | 100 | 0.92 |
| (applied to | | | | | | | |
| all biopsies) | | | | | | | |
| Eq (1) Y1 | 1 | 6/49 (12) | 100 | 83 | 68 | 100 | 0.91 |
| (applied to | | | | | | | |
| 49 biopsies) | | | | | | | |

¹Thresholds and models developed from the 49 cases taken at biopsy, and excluded repeat biopsies.
²With this value or greater classify as moderate-severe interstitial inflammation.

uHepcidin was selected because a non-biased proteomic approach showed that it was differentially expressed in the urine during the evolution of LN flares.

Immunohistochemical staining demonstrated that infiltrating interstitial leukocytes expressed Hepcidin in LN kidney biopsies, and human monocytes were shown to produce Hepcidin in response to treatment with interleukin-6 and interferon-α. uHepcidin was also significantly increased in patients with moderate-severe interstitial inflammation as compared to patients with mild or no inflammation (FIG. 1B). When used alone to classify the severity of interstitial inflammation, uHepcidin misclassified 22 of 64 biopsies (Table 3).

LFABP is made by the proximal tubule in response to injury, and was believed to be responsive to interstitial inflammation. Although it was significantly increased in the urine of patients with moderate-severe interstitial inflammation, there was less difference compared to mild and no inflammation than with uMCP-1 or uLFABP (FIG. 1B). uLFABP misclassified 14 of 64 biopsies (Table 3).

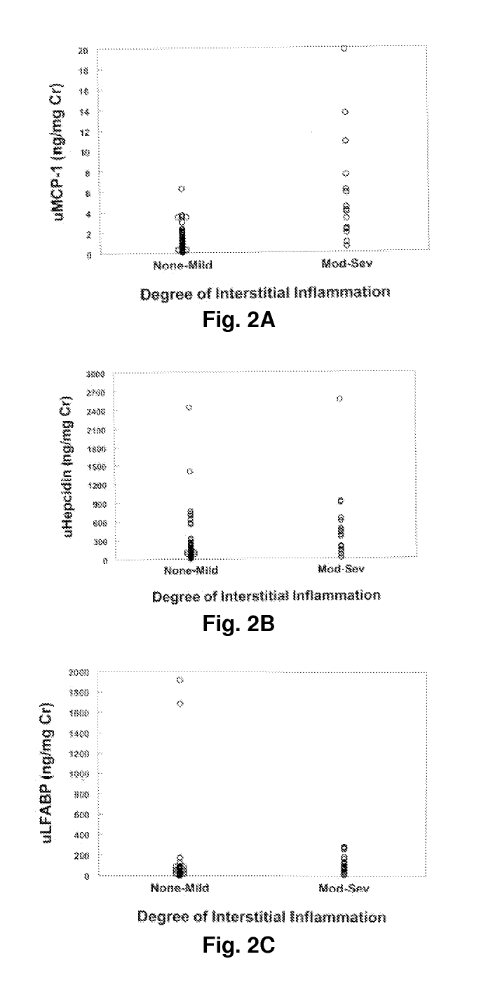

Table 3 lists the sensitivity, specificity, positive and negative predictive values of each of these individual biomarkers as predictors of the degree of interstitial inflammation. uMCP-1 performs fairly well, but uHepcidin and uLFABP do not. When individual data for each biomarker are examined it is apparent that there is considerable overlap of values in patients with no or mild inflammation and patients with moderate-severe inflammation (FIG. 2). This contributes to misclassifications and poor performance characteristics.

Combining Urine and Clinical Biomarkers

It was then determined if the performance characteristics to differentiate interstitial inflammation status could be improved, and misclassifications could be attenuated by combining candidate urine biomarkers and clinical biomarkers. All combinations of uMCP-1, uHepcidin, uLFABP, serum creatinine (SCr) and proteinuria (expressed as urine protein:creatinine ratio-uPCR) were tested by linear discriminant analysis, a procedure that produces optimal weights for the log-transformed variables involved. The linear discriminant analysis was based on the 49 urine samples collected at the time of biopsy and did not include any repeat biopsies. One preferred combination is given below:

Y1=0.992*ln(uMCP1)+2.213*ln(Scr) Eq(1)

Here Y₁ is the linear discriminant score, and the Y₁ value that gave the maximum sum of sensitivity and specificity is 1. At and above this cut-off biopsies were assigned to moderate-severe interstitial inflammation; below this cut-off biopsies were assigned to no-mild interstitial inflammation. The same threshold value of 1 gave the best sum of sensitivity and specificity when applied to all 64 subjects and had the least misclassification probability (Table 3).

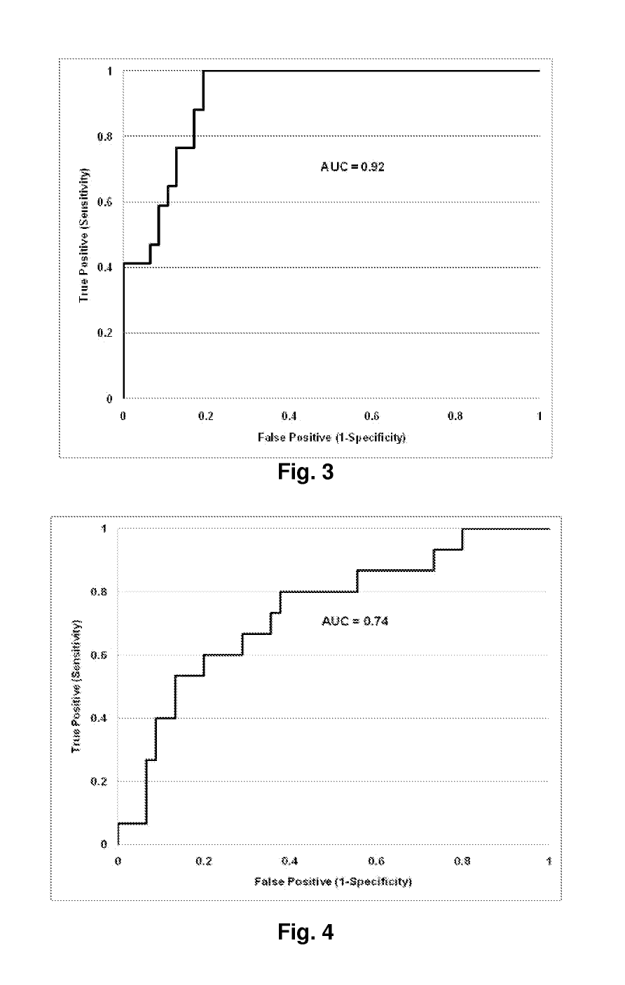

With this linear discriminant score only 9 of 64 biopsies (14%) were misclassified, specificity was 81% and sensitivity was 100% (Table 3). This means all misclassifications were from no-mild to moderate-severe inflammation. No cases of moderate-severe interstitial disease were misclassified. The positive predictive value was 67% and the negative predictive value was 100%. The receiver-operating characteristic (ROC) curve for this composite biomarker is shown in FIG. 3. The area under the curve (AUC) was 0.92.

| | Threshold | Misclassifications | Sens | Spec | PPV | NPV | AUC Under |
|---|---|---|---|---|---|---|---|
| Biomarker | Value | (%) | (%) | (%) | (%) | (%) | ROC |
| Eq (1) Y1 | 1 | 9/64 (14) | 100 | 81 | 67 | 100 | 0.92 |
| (applied to all | | | | | | | |
| LN biopsies) | | | | | | | |

For comparison all 5 variables were used to derive a biomarker from the same 49 cases. This composite biomarker had lower specificity and a higher number (11) of misclassified cases than Y1.

The misclassified patients could not be differentiated from correctly classified patients by the use of medications at the time of biopsy, including pulse methylprednisolone, oral corticosteroids or immunosuppressive drugs. Two misclassified patients received pulse methylprednisolone (22%), while 10 correctly classified patients received pulse corticosteroids (18%). The median dose of prednisone in the misclassified patients was 3 mg/d (range 0-60), and in the correctly classified patients it was 20 mg/d (range 0-60). Additionally, the misclassified patients could not be differentiated from correctly classified patients by the timing of their urine samples as only 2 gave urine samples after their biopsies.

Interestingly, the misclassified patients all had an elevated serum creatinine, and their average creatinine was significantly greater than the correctly classified patients (2.24±0.28 vs. 1.37±0.16 mg/dl, p=0.003). It is not likely that this finding can be used to identify patients that are likely to be misclassified as several correctly classified patients had serum creatinine values in this range or higher.

### Example 2

### A Biomarker for Interstitial Fibrosis

It was then determined if the urine and clinical biomarkers could be combined to yield a linear discriminant equation for interstitial fibrosis, as interstitial inflammation leads to injury that may result in interstitial fibrosis and chronic kidney disease. The ability to classify interstitial fibrosis as moderate-severe or none-mild was examined using discriminators based on urines obtained at biopsy (no repeat biopsies). For fibrosis, the best discriminant function is given by equation 2 with a threshold value of −1:

Y2=4.177*ln(uPCR)−1.425*ln(uHEP) Eq.2

The performance characteristics of individual biomarkers and the combined biomarker Y2 are given in Table 4 for all of the biopsies.

TABLE 4
Performance Characteristics of Biomarkers of
Interstitial Fibrosis for all the Biopsies1

| | Threshold | Misclassifications | Sens | Spec | PPV | NPV | AUC Under |
|---|---|---|---|---|---|---|---|
| Biomarker | Value² | (%) | (%) | (%) | (%) | (%) | ROC |
| uMCP-1 | 1.51 | 25/63(40) | 65 | 59 | 37 | 80 | 0.66 |
| uLFABP | 148 | 17/63(27) | 41 | 85 | 50 | 80 | 0.80 |
| uHepcidin | 32.5 | 14/63(22) | 35 | 93 | 67 | 80 | 0.48 |
| uPCR | 4.0 | 13/60(22) | 67 | 80 | 58 | 88 | 0.72 |
| Scr | 0.81 | 26/63(41) | 94 | 46 | 39 | 95 | 0.75 |
| Eq (2) Y2 | −1 | 13/60(22)³ | 53 | 87 | 57 | 85 | 0.74 |
| (applied to | | | | | | | |
| all biopsies) | | | | | | | |
| Eq (2) Y2 | −1 | 8/46(17) | 64 | 89 | 64 | 88 | 0.76 |
| (applied to | | | | | | | |
| 49 biopsies) | | | | | | | |

1Thresholds and models developed from the 46 cases taken at biopsy, and excluded repeat biopsies.
Note that in this set there are 46 cases instead of 49 as for Table 3 because 3 cases were missing uPCR.
²With this value or greater classify as moderate-severe interstitial fibrosis.
³There are 60 biopsies here instead of 64 as in Table 3 because of missing uPCR in 4 cases.

The combined biomarker Y2 threshold of −1 based on 46 biopsies did not produce the best sum of sensitivity and specificity for all 60 biopsies, but yielded the lowest misclassification proportion. The best sum of sensitivity and specificity was achieved with a threshold value of −2.94 (sensitivity 80%; specificity 62%) but misclassified 20 out of 61 cases (or 33%). The difference in the sum of sensitivity and specificity however is just 2%. The threshold Y2 value of −1 was thus favored given the lower rate of misclassification.

FIG. 4 shows the receiver-operating characteristic (ROC) curve for a composite biomarker of renal interstitial fibrosis. This ROC curve is based on Equation (2) which combines uHep, and uPCR to differentiate biopsies with no-mild interstitial fibrosis from moderate to severe interstitial fibrosis. The area under the curve is 0.74.

| | Threshold | Misclassifications | Sens | Spec | PPV | NPV | AUC Under |
|---|---|---|---|---|---|---|---|
| Biomarker | Value² | (%) | (%) | (%) | (%) | (%) | ROC |
| Eq (2) Y2 | −1 | 13/60(22) | 53 | 87 | 57 | 85 | 0.74 |
| (applied to all | | | | | | | |
| biopsies) | | | | | | | |

### Discussion of Example 1 and Example 2

The method described herein to non-invasively monitor changes in kidney pathology during the treatment of LN provides an important step forward in improving disease management and outcome.

In particular, a composite biomarker, uMCP1+SCr, accurately reflects renal interstitial inflammation in a moderately-sized cohort of SLE patients. Although individual candidate urine biomarkers were, on average, differentially expressed relative to the level of interstitial inflammation in a population, there was significant overlap among cases with and without interstitial inflammation, and this attenuated the performance of single urine proteins as biomarkers.

Equation (1) correctly classified 86% of the biopsies. Although the kidney biopsy is the gold-standard comparator for Equation (1), there is a finite rate of misclassification with tissue readings. The accuracy of a kidney biopsy depends on the size of the tissue sample obtained. For example, the correct diagnosis of glomerular disease or kidney allograft rejection requires an adequate biopsy defined by a minimum number of glomeruli and blood vessels. There is no information on correct classification of tubulointerstitial lesions by biopsy in SLE, however in a study of paired kidney transplant biopsies, interstitial fibrosis identified on the first biopsy was not seen in 12% of second biopsies. Because it was not felt that regression of fibrosis had occurred, this was thought to be an estimate of misclassification of tubulointerstitial disease by biopsy, and is close to that of our composite biomarker.

Urine biomarkers are thus less likely to misclassify kidney pathology because they reflect the total renal environment and are not subject to biopsy sampling errors and size variations.

In addition to using biomarker of interstitial inflammation for the evaluation of LN, such biomarker/s are useful to describe the renal interstitium in other types of kidney disease. This is relevant because tubulointerstitial injury, including interstitial inflammation and fibrosis is a risk factor for renal functional decline and poor response to therapy in a variety of disorders. These include membranous nephropathy, focal segmental glomerulosclerosis, IgA nephropathy, diabetic nephropathy, and renal transplant failure. Similar to LN, interstitial inflammation appears to be a precursor to interstitial fibrosis in these diseases.

Thus, Example 2 show that combinations of urine proteins and clinical variables are useful to derive useful composite biomarkers that reflect specific pathologic lesions in the kidneys of patients with LN.

### Methods for Example 1 and Example 2

### Kidney Biopsy Cohort

The cohort was comprised of 64 kidney biopsies from 61 patients, all of whom had at least 4 American College of Rheumatology criteria for systemic lupus erythematosus (SLE), including immune-complex glomerulonephritis, and many of whom participated in the Ohio SLE Study. Three patients had repeat biopsies. Urine was collected on the day of biopsy or within 24 hours, except in 12 cases where urine was collected within 2 (n=4), 3 (n=2), 4, 6, 7 (n=2), 12, and 13 days of kidney biopsy. After urine was collected it was centrifuged to remove sediment and stored in preservative-free aliquots at −80° C. until use.

Measurement of Interstitial Inflammation and Fibrosis in Kidney Biopsies

Interstitial inflammation and interstitial fibrosis were semi-quantitatively graded as none, mild, moderate, or severe on light-microscopic sections for clinical biopsy reports by a nephro-pathologist blinded to urine biomarker data. The stains used to estimate the percentage of involved cortex were hematoxylin and eosin, periodic-acid Schiff, and tri-chrome. None was considered to be up to 5% of the renal interstitium; mild between 6 and 25%, moderate between 26 and 50%, and severe greater than 50%. For analysis biopsies with no-mild inflammation were combined, and biopsies with moderate and severe fibrosis were combined. The rationale for this grouping was to model and distinguish clinically significant interstitial disease.

Measurement of Urine Biomarkers

Urine MCP1 levels were measured using the Quantikine Human CCL2/MCP1 ELISA kit from R &D Systems (Minneapolis, Minn.). uMCP-1 was normalized to urine creatinine. Creatinine was measured with a Creatinine Detection Kit (Assay Designs, Ann Arbor, Mich.). The final values were expressed as ng MCP-1/mg creatine.

Urine L-FABP level was measured using the Human L-FABP ELISA kit from CMIC Ltd. (Tokyo, Japan) following the manufacture's protocol and uLFABP was corrected by urine creatinine. The final values were expressed as ng L-FABP/mg creatine.

Hepcidin-25 was measured by EIA (Bachem Group, Torrance, Calif.). The hepcidin-25 standard Liver-Expressed Antimicrobial Peptide 1 (LEAP1) from Peptides International Inc (Louisville, Ky.) was used to validate this EIA. The R-squared value was 0.9967 for LEAP1 from 0-50 ng/ml using sigmoid regression. The coefficient of variation (CV) for a fixed hepcidin-25 concentration of 1.56 ng/ml was 3.49% intra-assay and 3.43% inter-assay. Urine Hepcidin were then normalized to urine creatinine with the final value expressed as ng Hepcidin/mg creatinine.

Data Analysis

Fisher's linear discriminant analysis was used to determine the discriminant score function based on one or more normally distributed components. The procedure produces an optimally weighted linear function of the chosen log-transformed markers and the discriminating threshold value minimizes the expected number of misclassifications under the normal model. This does not necessarily maximize the sum of sensitivity and specificity. We modify the threshold value to be the one that maximizes this sum for the observed data. The data were log-transformed because this gave a good fit to a normal distribution. The software used for analysis was SAS JMP 9.0 (Cary, N.C.).

Comparisons of two groups were done by the Mann-Whitney test. A two-tailed P<0.05 was considered significant.

### Example 3

Application to Other Types of Kidney/Glomerular Disease

The interstitial inflammation biomarker equation 1 was applied to 10 biopsies that were not LN. These biopsies included an idiopathic immune-complex glomerulonephritis (1), pauci-immune necrotizing and crescentic glomerulonephritis (1), membranous glomerulopathy (1), diabetic glomerulosclerosis (1), IgA nephropathy (1), advanced chronic kidney disease (1), glomerular basement membrane abnormalities (2), non-specific findings (2). Only one of these biopsies had moderate-severe interstitial inflammation, the rest had none-mild.

Equation 1 correctly classified 8 of the 10 biopsies, including the biopsy with severe interstitial inflammation. Two biopsies with no-mild interstitial inflammation were misclassified as moderate-severe, and like the misclassified LN patients described previously, these patients had elevated serum creatinine levels.

In another specific embodiment, there is provided a method for evaluating renal status in a subject, comprising: performing one or more assays configured to detect a kidney injury marker selected from at least two markers are selected from the group consisting of: urine monocyte chemotactic protein-1 (uMCP-1), serum creatinine (Src), hepcidin (uHep), proteinura expressed as a ratio of urine protein:creatine (uPCR), and urine liver-type fatty acid binding protein (uLFABP), on a body fluid sample obtained from the subject to provide one or more assay results; and correlating the assay result(s) to the renal status of the subject

In certain embodiments, the correlation step comprises correlating the assay result(s) to one or more of risk stratification, diagnosis, staging, prognosis, classifying and monitoring of the renal status of the subject.

In certain embodiments, the correlating step comprises assigning a likelihood of one or more current changes in renal status to the subject based on the assay result(s).

In certain embodiments, the one or more current changes in renal status comprise one or more of: interstitial inflammation and interstitial fibrosis.

In certain embodiments, the correlating step comprises assigning a diagnosis of the occurrence or nonoccurrence of one or more of: interstitial inflammation and interstitial fibrosis, to the subject based on the assay result(s).

In certain embodiments, the method is a method of diagnosing the occurrence or nonoccurrence of an injury to, or reduced, renal function in the subject.

In certain embodiments, the method is a method of assigning a risk of the future occurrence or nonoccurrence of an injury to, or reduced, renal function in the subject.

In certain embodiments, the one or more changes in renal status comprise one or more of injury to, or reduced, renal function in the subject within 72 hours of the time at which the body fluid sample is obtained.

In certain embodiments, the one or more changes in renal status comprise one or more of injury to, or reduced, renal function in the subject within 48 hours of the time at which the body fluid sample is obtained.

In certain embodiments, the one or more changes in renal status comprise one or more of injury to, or reduced, renal function in the subject within 24 hours of the time at which the body fluid sample is obtained.

In certain embodiments, the one or more changes in renal status comprise one or more of injury to, or reduced, renal function in the subject within 2 hours of the time at which the body fluid sample is obtained.

In certain embodiments, the one or more changes in renal status comprise one or more of injury to, or reduced, renal function in the subject substantially at the time at which the body fluid sample is obtained.

### Example 4

### Biomarkers and Uses Thereof

In another embodiment, there is provided that a biomarker to predict one or more of lupus nephritis, renal fibrosis and chronic kidney disease, consisting two or more markers selected from: urine monocyte chemotactic protein-1 (uMCP-1), urine hepcidin (uHep), urine liver-type fatty acid binding protein (uLFABP), serum creatinine (Scr) and proteinura, expressed as a ratio of urine protein:creatine (uPCR).

In certain embodiments, there is provided a biomarker of interstitial inflammation in lupus nephritis (LN), comprising: urine monocyte chemotactic protein-1 (uMCP-1) and serum creatinine (Scr).

In certain embodiments, there is provided a biomarker of interstitial fibrosis in lupus nephritis (LN), comprising: urine hepcidin (uHep) and proteinura, expressed as a ratio of urine protein:creatine (uPCR).

In another embodiment, there is provided the use of two or more kidney injury markers selected from the group consisting of: urine monocyte chemotactic protein-1 (uMCP-1), serum creatinine (Src), hepcidin (uHep), proteinura expressed as a ratio of urine protein:creatine (uPCR), and urine liver-type fatty acid binding protein (uLFABP), for the evaluation of renal injury.

In another embodiment, there is provided the of two or more kidney injury markers selected from the group consisting of: urine monocyte chemotactic protein-1 (uMCP-1), serum creatinine (Src), hepcidin (uHep), proteinura expressed as a ratio of urine protein:creatine (uPCR), and urine liver-type fatty acid binding protein (uLFABP), for the evaluation of acute renal injury.

In another embodiment, there is provided the of kidney injury markers: urine monocyte chemotactic protein-1 (uMCP-1) and serum creatinine (Src) for the evaluation of interstitial inflammation in lupus nephritis.

In another embodiment, there is provided the of kidney injury markers: hepcidin (uHep) and proteinura, expressed as a ratio of urine protein:creatine (uPCR), for the evaluation of interstitial fibrosis in lupus nephritis.

### Example 5

In another aspect, there is provided herein method for evaluating renal status in a human test subject. In one embodiment, the method comprises:

a) measuring a level of expression in a sample of the test subject of at least two markers selected from the group consisting of: urine monocyte chemotactic protein-1 (uMCP-1), serum creatinine (Src), hepcidin (uHep), proteinura expressed as a ratio of urine protein:creatine (uPCR), and urine liver-type fatty acid binding protein (uLFABP), thereby obtaining a sample dataset; and

b) applying a classifier to the sample dataset to thereby classify the test subject into a class representing human subjects having interstitial nephritis and/or interstitial fibrosis or a class representing human subjects not having interstitial nephritis and/or interstitial fibrosis.

The classifier is able to discriminate between human subjects having interstitial nephritis and/or interstitial fibrosis and human subjects not having interstitial nephritis and/or interstitial fibrosis. The classifier is derived from data representing a level of expression of at least two markers in samples of human subjects having interstitial nephritis and/or interstitial fibrosis and in samples of human subjects not having interstitial nephritis and/or interstitial fibrosis, thereby evaluating renal status in a human test subject.

In certain embodiments, applying the classifier to the sample dataset comprises using a computer programmed to apply the classifier to a dataset representing a level of expression of each marker in a sample of a human individual to thereby classify the human individual into the class representing human subjects having interstitial nephritis and/or interstitial fibrosis or the class representing human subjects not having interstitial nephritis and/or interstitial fibrosis.

In another aspect, there is provided herein a method for evaluating renal status in a human test subject, where the method comprises:

a) obtaining a sample dataset representing a level of expression in a sample of the test subject of subject of at least two markers selected from the group consisting of: urine monocyte chemotactic protein-1 (uMCP-1), serum creatinine (Src), hepcidin (uHep), proteinura expressed as a ratio of urine protein:creatine (uPCR), and urine liver-type fatty acid binding protein (uLFABP); and

b) using a computer, applying a classifier to the sample dataset to thereby classify the test subject into a class representing human subjects having interstitial nephritis and/or interstitial fibrosis or a class representing human subjects not having interstitial nephritis or interstitial fibrosis.

The classifier is able to discriminate between human subjects having interstitial nephritis and/or interstitial fibrosis and human subjects not having interstitial nephritis and/or interstitial fibrosis. The classifier is derived from data representing a level of expression of each marker of the marker set in samples of human subjects having interstitial nephritis and/or interstitial fibrosis and in samples of human subjects not having interstitial nephritis and/or interstitial fibrosis.

The computer is programmed to apply the classifier to a dataset representing a level of expression of marker in a sample of a human individual to thereby classify the test individual into the class representing human subjects having interstitial nephritis fibrosis and/or interstitial fibrosis or the class representing human subjects not having interstitial nephritis and/or interstitial fibrosis, thereby evaluating renal status in a human test subject.

In another aspect, there is provided herein a method for profiling gene expression in a human test subject, where the method comprises: using a computer, applying a classifier to a sample dataset representing a level of expression in a sample of the test subject of at least two markers selected from the group consisting of: urine monocyte chemotactic protein-1 (uMCP-1), serum creatinine (Src), hepcidin (uHep), proteinura expressed as a ratio of urine protein:creatine (uPCR), and urine liver-type fatty acid binding protein (uLFABP), to thereby classify the test subject into a class representing human subjects having interstitial nephritis and/or interstitial fibrosis or a class representing human subjects not having interstitial nephritis and/or interstitial fibrosis.

The classifier is able to discriminate between human subjects having interstitial nephritis and/or interstitial fibrosis and human subjects not having interstitial nephritis and/or interstitial fibrosis. The classifier is derived from data representing a level of expression of each marker in samples of human subjects having interstitial nephritis and/or interstitial fibrosis and in samples of human subjects not having interstitial nephritis and/or interstitial fibrosis.

The computer is programmed to apply the classifier to a dataset representing a level of expression of each marker in a sample of a human individual to thereby classify the human individual into the class representing human subjects having interstitial nephritis and/or interstitial fibrosis or the class representing human subjects not having interstitial nephritis and/or interstitial fibrosis, thereby evaluating renal status in a human test subject.

In certain embodiments, the method further comprises obtaining the sample dataset by measuring the level of expression of each marker in the sample of the test subject, prior to applying the classifier to the sample dataset.

In certain embodiments, the classifier is based on a linear Discriminant analysis equation.

In certain embodiments, the renal status being evaluated is interstitial nephritis, and the classifier has a format: Eq(1) Y1=0.992*ln(uMCP1)+2.213*ln(Scr), where

Y1, if ≧1, classifies the test subject into the class representing human subjects having moderate-severe interstitial nephritis;

Y1, if ≦1, classifies the test subject into the class representing human subjects not having interstitial nephritis or having mild interstitial nephritis;

uMPC1 represents the level of expression of urine monocyte chemoattractant protein-1 in the sample of the test subject; and

Scr represents the level of serum creatinine in the sample of the test subject.

In certain embodiments, the renal status being evaluated is interstitial fibrosis, and the classifier has a format: Eq.2 Y2=4.177*ln(uPCR)−1.425*ln(uHEP), where

Y2, if ≧−1, classifies the test subject into the class representing human subjects having moderate-severe interstitial fibrosis;

Y2, if ≦−1, classifies the test subject into the class representing human subjects not having interstitial fibrosis or mild interstitial fibrosis;

uHep represents the level of expression of urine hepcidin in the sample of the test subject; and

uPCR, proteinura expressed as a ratio of urine protein:creatine (uPCR), represents the level of serum creatinine in the sample of the test subject.

Generation of a Dataset

The quantitative data is obtained for each component of the dataset and inputted into an analytic process with previously defined parameters (the predictive model) and then used to generate a result.

The data may be obtained via any technique that results in an individual receiving data associated with a sample. For example, an individual may obtain the dataset by generating the dataset himself by methods known to those in the art. Alternatively, the dataset may be obtained by receiving the dataset from another individual or entity. For example, a laboratory professional may generate the dataset while another individual, such as a medical professional, or may input the dataset into an analytic process to generate the result. One of skill should understand that although reference is made to “a sample” throughout the specification that the quantitative data may be obtained from multiple samples varying in any number of characteristics, such as the method of procurement, time of procurement, tissue origin, etc.

Quantitative Data Regarding Markers

The quantitative data associated with the markers of interest can be any data that allows generation of a result useful for the classification, including measurement of DNA or RNA levels associated with the markers but is typically protein expression patterns. Protein levels can be measured via any method known to those of skill of art that generates a quantitative measurement either individually or via high-throughput methods as part of an expression profile. For example, a urine derived patient sample may be applied to a specific binding agent or panel of specific binding agents to determine the presence and quantity of the protein markers of interest.

Uses of Results Generated by Analytic Process

The datasets from containing quantitative data for components of the dataset are inputted into an analytic process and used to generate a result. The result can be any type of information useful for making a classification, a continuous variable, or a vector. For example, the value of a continuous variable or vector may be used to determine the likelihood that a sample is associated with a particular classification. The classification refer to any type of information or the generation of any type of information associated with a particular condition, for example, diagnosis, staging, assessing extent of progression, prognosis, monitoring, therapeutic response to treatments, screening to identify compounds that act via similar mechanisms as known treatments, prediction of interstitial inflammation (IN), stable vs. unstable, identifying complications of the disease

In a preferred embodiment, the result is used for diagnosis or detection of the occurrence of an interstitial inflammation (IN), particularly where such IN is indicative of a propensity for interstitial damage, which is generally preceded by interstitial inflammation, is the most important risk factor for developing chronic kidney disease and end stage kidney disease. But INF can be treated to prevent damage and chronic kidney disease, and end stage kidney disease.

In this embodiment, a reference or training set containing “healthy” and “SLE” samples is used to develop a predictive model. A dataset, preferably containing protein expression levels of markers indicative of the IN, is then inputted into the predictive model in order to generate a result. The result may classify the sample as either “healthy” or “IN”. In other embodiments, the result is a continuous variable providing information useful for classifying the sample, e.g., where a high value indicates a high probability of being an “IN” sample and a low value indicates a high probability of being a “healthy” sample.

Determination of Treatments

In other embodiments, the result is used determine response to IN treatments. In this embodiment, the reference or training dataset and the predictive model is the same as that used to diagnose (samples of from individuals with disease and those without). The dataset is composed of individuals with known disease and/or disease which have been administered a particular treatment and it is determined whether the samples trend toward or lie within a normal, healthy classification versus a disease classification.

In another embodiment, the result is used for drug screening, i.e., identifying compounds that act via similar mechanisms as known drug treatments. In this embodiment, a reference or training set containing individuals treated with a known drug treatment and those not treated with the particular treatment can be used develop a predictive model. A dataset from individuals treated with a compound with an unknown mechanism is input into the model. If the result indicates that the sample can be classified as coming from a subject dosed with a known drug treatment, then the new compound is likely to act via the same mechanism.

One of skill will also recognize that the results generated using these methods can be used in conjunction with any number of the various other methods known to those of skill in the art for diagnosing and monitoring the disease.

Analysis of Protein Forms

It is understood that proteins frequently exist in a sample in a plurality of different forms. When detecting or measuring a protein in a sample, the ability to differentiate between different forms of a protein depends upon the nature of the difference and the detection method that is used. In particular, a sandwich immunoassay, having two antibodies directed against different epitopes on a protein, is useful to detect all forms of the protein that contain both epitopes and will not detect those forms that contain only one of the epitopes. In certain embodiments, one or more forms of the urine protein could be better marker than certain other forms. In a particular embodiment, it is useful to employ an assay method that distinguishes between forms of a protein and that specifically detects and measures a desired form or forms of the protein.

Mass spectrometry is an especially useful method to distinguish between different forms of proteins since the different forms typically have different masses that can be resolved by mass spectrometry. Various forms of mass spectrometry are useful for detecting the protein forms, including laser desorption approaches, such as SELDI.

Thus, in certain embodiments, when reference is made herein to detecting a particular protein or to measuring the amount of a particular protein, it means detecting and measuring the protein and resolving various forms of protein.

Biochips

In one embodiment, a sample is analyzed by means of a biochip. A biochip generally comprises a solid substrate having a substantially planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), and others.

One useful mass spectrometric technique for use in the invention is “Surface Enhanced Laser Desorption and Ionization” or “SELDI,” which is a method of desorption/ionization gas phase ion spectrometry (e.g., mass spectrometry) in which an analyte (here, one or more of the biomarkers) is captured on the surface of a SELDI mass spectrometry probe.

Test Devices and Kits

In another aspect, there is provided a test device that includes a test surface comprising a plurality of discrete addressable locations corresponding to the subject-derived markers, where each the location comprising an antibody immobilized at the location selected to bind for detection one of the subject-derived markers.

In another aspect, there is provided a kit for qualifying impending flare-ups of systemic lupus erythematosus status. The kits are useful to detect the markers. The kit can include a solid support, such as a chip, a microtiter plate or a bead or resin having a capture reagent attached thereon, where the capture reagent binds the markers. The kit can comprise probes for ELISA, mass spectrometry probes for SELDI, such as ProteinChip® arrays. The kit can also include a solid support with a reactive surface, and a container comprising the biospecific capture reagent. The kit can also include a washing solution or instructions for making a washing solution, such that the combination of the capture reagent and the washing solution allows capture of the biomarker or biomarkers on the solid support for subsequent detection by, e.g., mass spectrometry. The kit may include more than type of adsorbent, each present on a different solid support. The kit can also include instructions that may inform a consumer about how to collect the sample, how to wash the probe or the particular markers to be detected. In yet another embodiment, the kit can include one or more containers with the marker samples, to be used as standard(s) for calibration.

Thus, in a particular embodiment, the kit is especially useful for detecting an impending kidney nephritis flare-up episode characterized by non-physiological levels of a panel of urine protein markers. In one non-limiting example, the kit can include anti-MCP-1, anti-Scr, anti-Hep and/or anti-PCR antibodies or fragments thereof that specifically bind to one or more epitopes of the urine protein markers, and a reagent that binds directly or indirectly to the antibody or fragment thereof.

Methods of Following Response Over Time and/or Predicting Response to Therapeutic Agents

In another aspect, there is provided herein a method of following the response of the patient over time, and/or predicting the response of a patient to treatment with a therapeutic agent. The method comprises contacting a sample obtained from the patient to measure the levels of expression of two or more of the markers described herein.

The expression levels are then used to provide an expression profile for the patient that is then compared to the drug-gene correlations, wherein a positive correlation between a drug and expressed levels of the markers in the patient indicates that the patient would be sensitive to the drug, and wherein a negative correlation between a drug and the expressed levels in the patient indicates that the patient would not be responsive to the drug.

In some embodiments, the effectiveness of the agent's ability to alter chemosensitivity can be tested using standard assays. The agent is tested by conducting assays in that sample are co treated with the newly identified agent along with a previously known therapeutic agent. The choice of previously known therapeutic agent is determined based upon the gene-drug correlation between the gene or genes whose expression is affected by the new agent.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

The citation of any reference herein is not an admission that such reference is available as prior art to the instant invention. Any publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A biomarker to predict one or more of lupus nephritis, renal fibrosis and chronic kidney disease, consisting two or more markers selected from: urine monocyte chemotactic protein-1 (uMCP-1), urine hepcidin (uHep), serum creatinine (Scr) and proteinura, expressed as a ratio of urine protein:creatine (uPCR).

2. A biomarker of interstitial inflammation in lupus nephritis (LN), comprising: urine monocyte chemotactic protein-1 (uMCP-1) and serum creatinine (Scr).

3. A biomarker of interstitial fibrosis in lupus nephritis (LN), comprising: urine hepcidin (uHep) and proteinura, expressed as a ratio of urine protein:creatine (uPCR).

4. A method for generating a result useful in diagnosing and non-invasively monitoring renal pathology using samples obtained from a mammalian subject, comprising:
obtaining a dataset associated with the samples, wherein the dataset comprises protein expression levels for at least two markers selected from the group consisting of:
urine monocyte chemotactic protein-1 (uMCP-1), serum creatinine (Src), hepcidin (uHep), and proteinura expressed as a ratio of urine protein:creatine (uPCR);
and
inputting the dataset into an analytical process that uses the data to generate a result useful in diagnosing and monitoring the renal pathology.

5. (canceled)

6. The method of claim 4, wherein the samples comprise urine and serum obtained from the subject at substantially the same time.

7. The method of claim 4, wherein the kidney pathology comprises one or more of: glomerular diseases; systemic lupus erythematosus (SLE) disease; interstitial inflammation in lupus nephritis (LN); interstitial fibrosis in lupus nephritis (LN); renal-interstitial inflammation (INF); idiopathic immune-complex glomerulonephritis; pauci-immune necrotizing and crescentic glomerulonephritis; membranous glomerulopathy; diabetic glomerulosclerosis; IgA nephropathy; advanced chronic kidney disease; and glomerular basement membrane abnormalities.

8. The method of claim 4, wherein the analytical process is a Linear Discriminant Analysis model.

9. The method of claim 4, wherein the analytical process comprises use of a predictive model.

10. The method of claim 4, wherein the analytical process comprises comparing the obtained dataset with a reference dataset.

11. The method of claim 4, wherein the reference dataset comprises protein expression levels obtained from one or more healthy control subjects, or comprises protein expression levels obtained from one or more subjects diagnosed with renal-interstitial inflammation (INF).

12. The method of claim 4, further comprising obtaining a statistical measure of a similarity of the obtained dataset to the reference dataset.

13. A method for classifying a sample obtained from a mammalian subject, comprising:
obtaining a dataset associated with the sample,
wherein the dataset comprises expression levels for at least two markers selected from the group consisting of:
urine monocyte chemotactic protein-1 (uMCP-1), serum creatinine (Src), hepcidin (uHep), and proteinura expressed as a ratio of urine protein:creatine (uPCR);
inputting the dataset into an analytical process that uses the data to classify the sample, wherein the classification is selected from the group consisting of a lupus nephritis classification, a healthy classification, a renal-interstitial inflammation classification, a no renal-interstitial inflammation classification, a medication exposure classification, a no medication exposure classification;
and
classifying the sample according to the output of the process.

14. The method of claim 13, wherein the analytical process comprises use of a predictive model.

15. The method of claim 13, wherein the analytical process comprises comparing the obtained dataset with a reference dataset.

16. The method of claim 13, wherein the reference dataset comprises protein expression levels obtained from one or more healthy control subjects, or comprises protein expression levels obtained from one or more subjects diagnosed with a renal-interstitial inflammation (INF).

17. A method for classifying a sample obtained from a mammalian subject, comprising:
obtaining a dataset associated with the sample,
wherein the dataset comprises expression levels for at least two markers selected from the group consisting of:
urine monocyte chemotactic protein-1 (uMCP-1), serum creatinine (Src), hepcidin (uHep), and proteinura expressed as a ratio of urine protein:creatine (uPCR);
inputting the data into a predictive model that uses the data to classify the sample, wherein the classification is selected from the group consisting of: a renal-interstitial inflammation classification, a no renal-interstitial inflammation classification, wherein the predictive model has at least one quality metric of at least 0.7 for classification;
and,
classifying the sample according to the output of the predictive model.

18. The method of claim 17, wherein the predictive model has a quality metric of at least 0.8 for classification.

19. The method of claim 17, wherein the predictive model has a quality metric of at least 0.9 for classification.

20. The method of claim 18, wherein the quality metric is selected from area-under-curve (AUC) and accuracy.

21. The method of claim 17, wherein the limits of the predictive model are adjusted to provide at least one of sensitivity or specificity of at least 0.7.

22. The method of claim 17, wherein the limits of the predictive model are adjusted to provide at least one of sensitivity or specificity of at least 0.9.

23. The method of claim 17, further comprising using the classification for diagnosis, staging, prognosis, kidney inflammation levels, assessing extent of progression, monitoring a therapeutic response, predicting a renal-interstitial inflammation (INF) episode, or distinguishing stable from unstable manifestations of renal-interstitial inflammation (INF).

24. The method of claim 17, wherein the dataset further comprises quantitative data for one or more clinical indications.

25. The method of claim 17, wherein the analytic process comprises using a Linear Discriminant Analysis model.

26. The method of claim 17, wherein the process comprises using a Linear Discriminant Analysis model or a Logistic Regression model, and the model comprises terms selected to provide a quality metric greater than 0.75.

27. The method of claim 4, further comprising obtaining a plurality of classifications for a plurality of samples obtained at a plurality of different times from the subject.

28. A method of analyzing a subject sample for one or more subject-derived markers selected to identify at least a beginning of a renal-interstitial inflammation (INF) and/or tubulointerstitial inflammation (TI) episode in patients with lupus nephritis (LN), comprising:
assaying the sample for the presence or amount of subject-derived markers related to a INT or TI episode, wherein at least two markers are selected from the group consisting of:
urine monocyte chemotactic protein-1 (uMCP-1), serum creatinine (Src), hepcidin (uHep), and proteinura expressed as a ratio of urine protein:creatine (uPCR);
and
characterizing the subject's risk of having, or at risk for having, the INF and/or TI episode based upon the presence or amount of the markers.

29. A method for assigning a therapy regimen and/or assigning a prognosis to a subject diagnosed with or suspected of suffering from an interstitial inflammation episode, comprising:
performing an assay on a sample obtained from the subject, wherein the assay provides one or more detectable signals related to the presence or amount of one or more subject-derived markers independently selected from the group consisting of markers related to kidney flare episodes, or markers related to the subject-derived markers;
wherein at least two markers are selected from the group consisting of:
urine monocyte chemotactic protein-1 (uMCP-1), serum creatinine (Src), hepcidin (uHep), and proteinura expressed as a ratio of urine protein:creatine (uPCR);
and
correlating the signal(s) obtained from the assay method to ruling in or out a therapy regimen for the subject and/or assigning a prognosis to the subject.

30. The method of claim 29, wherein the markers consist of: urine monocyte chemotactic protein-1 (uMCP-1) and serum creatinine (Src).

31. A method for assigning a therapy regimen and/or assigning a prognosis to a subject diagnosed with or suspected of suffering from interstitial fibrosis, comprising:
performing an assay on a sample obtained from the subject, wherein the assay provides one or more detectable signals related to the presence or amount of one or more subject-derived markers independently selected from the group consisting of markers related to kidney flare episodes, or markers related to the subject-derived markers;
wherein at least two markers are selected from the group consisting of:
urine monocyte chemotactic protein-1 (uMCP-1), serum creatinine (Src), hepcidin (uHep), and proteinura expressed as a ratio of urine protein:creatine (uPCR);
and
correlating the signal(s) obtained from the assay method to ruling in or out a therapy regimen for the subject and/or assigning a prognosis to the subject.

32. The method of claim 31, wherein the markers consist of: hepcidin (uHep) and proteinura expressed as a ratio of urine protein:creatine (uPCR).

33. A method of claim 29, wherein the method rules in or out an assignment of the subject to early goal-directed therapy.

34. A method of claim 29, wherein the correlating step comprises comparing one or more subject-derived marker concentrations to a predetermined threshold level for a particular marker of interest.

35. A method of claim 29, wherein the correlating step comprises:
determining the concentration of the subject-derived markers,
calculating a single response value based on the concentration of the subject-derived markers, and
comparing the response value to one or more predetermined threshold levels for the response value.

36. A method of claim 29, wherein the correlating step comprises:
comparing the subject-derived marker concentrations to a predetermined threshold level for a particular marker of interest and determining the concentration of the subject-derived markers,
calculating a single response value based on the concentration of each of the subject-derived markers, and
comparing the response value to a predetermined threshold level for the panel response value.

37. A method of claim 4, wherein the sample is from a human.

38. A method of claim 4, wherein the assay method comprises an immunoassay.

39. A method of claim 4, wherein the method rules in or out one or more treatments for inclusion in a therapy regimen comprising administration of immunosuppressive therapy.

40. A method for diagnosing a disease condition characterized by altered levels of at least two markers selected from the group consisting of:
urine monocyte chemotactic protein-1 (uMCP-1), serum creatinine (Src), hepcidin (uHep), and proteinura expressed as a ratio of urine protein:creatine (uPCR);
the method comprising:
contacting a sample from a subject with an antibody or fragment thereof that specifically binds to one or more binding sites on the marker, and
quantifying the marker levels in the sample; wherein the altered levels of the markers is indicative of the disease condition.

41. The method of claim 40, wherein the antibody specifically binds an epitope contained within the marker.

42. A kit for detecting a disease condition characterized by non-physiological levels of at least two markers are selected from the group consisting of:
urine monocyte chemotactic protein-1 (uMCP-1), serum creatinine (Src), hepcidin (uHep), and proteinura expressed as a ratio of urine protein:creatine (uPCR);
the kit comprising: an anti-marker antibody or fragment thereof that specifically binds to the marker, and a reagent that binds directly or indirectly to the antibody or fragment thereof.

43. The kit of claim 42, wherein the anti-marker antibody or fragment thereof is immobilized on a support.

44. A method for evaluating renal status in a subject, comprising:
performing one or more assays configured to detect a kidney injury marker selected from at least two markers are selected from the group consisting of: urine monocyte chemotactic protein-1 (uMCP-1), serum creatinine (Src), hepcidin (uHep), and proteinura expressed as a ratio of urine protein:creatine (uPCR), on a body fluid sample obtained from the subject to provide one or more assay results; and
correlating the assay result(s) to the renal status of the subject

45. A method of claim 44, wherein the correlation step comprises correlating the assay result(s) to one or more of risk stratification, diagnosis, staging, prognosis, classifying and monitoring of the renal status of the subject.

46. A method of claim 44, wherein the correlating step comprises assigning a likelihood of one or more current changes in renal status to the subject based on the assay result(s).

47. A method of claim 46, wherein the one or more current changes in renal status comprise one or more of: interstitial inflammation and interstitial fibrosis.

48. A method of claim 44, wherein the correlating step comprises assigning a diagnosis of the occurrence or nonoccurrence of one or more of: interstitial inflammation and interstitial fibrosis, to the subject based on the assay result(s).

49. A method of claim 44, wherein the method is a method of diagnosing the occurrence or nonoccurrence of an injury to, or reduced, renal function in the subject.

50. A method of claim 44, wherein the method is a method of assigning a risk of the future occurrence or nonoccurrence of an injury to, or reduced, renal function in the subject.

51. A method of claim 44, wherein the one or more changes in renal status comprise one or more of injury to, or reduced, renal function in the subject within 72 hours of the time at which the body fluid sample is obtained.

52. A method of claim 44, wherein the one or more changes in renal status comprise one or more of injury to, or reduced, renal function in the subject within 48 hours of the time at which the body fluid sample is obtained.

53. A method of claim 44, wherein the one or more changes in renal status comprise one or more of injury to, or reduced, renal function in the subject within 24 hours of the time at which the body fluid sample is obtained.

54. A method of claim 44, wherein the one or more changes in renal status comprise one or more of injury to, or reduced, renal function in the subject within 2 hours of the time at which the body fluid sample is obtained.

55. A method of claim 44, wherein the one or more changes in renal status comprise one or more of injury to, or reduced, renal function in the subject substantially at the time at which the body fluid sample is obtained.

56. (canceled)

57. (canceled)

58. (canceled)

59. (canceled)

60. A method for evaluating renal status in a human test subject, the method comprising:
a) measuring a level of expression in a sample of the test subject of at least two markers selected from the group consisting of: urine monocyte chemotactic protein-1 (uMCP-1), serum creatinine (Src), hepcidin (uHep), and proteinura expressed as a ratio of urine protein:creatine (uPCR), thereby obtaining a sample dataset;
and
b) applying a classifier to the sample dataset to thereby classify the test subject into a class representing human subjects having interstitial nephritis and/or interstitial fibrosis or a class representing human subjects not having interstitial nephritis and/or interstitial fibrosis,
wherein the classifier is able to discriminate between human subjects having interstitial nephritis and/or interstitial fibrosis and human subjects not having interstitial nephritis and/or interstitial fibrosis, and
wherein the classifier is derived from data representing a level of expression of at least two markers in samples of human subjects having interstitial nephritis and/or interstitial fibrosis and in samples of human subjects not having interstitial nephritis and/or interstitial fibrosis, thereby evaluating renal status in a human test subject.

61. The method of claim 62, wherein the applying the classifier to the sample dataset comprises using a computer programmed to apply the classifier to a dataset representing a level of expression of each marker in a sample of a human individual to thereby classify the human individual into the class representing human subjects having interstitial nephritis and/or interstitial fibrosis or the class representing human subjects not having interstitial nephritis and/or interstitial fibrosis.

62. A method for evaluating renal status in a human test subject, the method comprising:
a) obtaining a sample dataset representing a level of expression in a sample of the test subject of subject of at least two markers selected from the group consisting of: urine monocyte chemotactic protein-1 (uMCP-1), serum creatinine (Src), hepcidin (uHep), and proteinura expressed as a ratio of urine protein:creatine (uPCR);
and
b) using a computer, applying a classifier to the sample dataset to thereby classify the test subject into a class representing human subjects having interstitial nephritis and/or interstitial fibrosis or a class representing human subjects not having interstitial nephritis or interstitial fibrosis,
wherein the classifier is able to discriminate between human subjects having interstitial nephritis and/or interstitial fibrosis and human subjects not having interstitial nephritis and/or interstitial fibrosis,
wherein the classifier is derived from data representing a level of expression of each marker of the marker set in samples of human subjects having interstitial nephritis and/or interstitial fibrosis and in samples of human subjects not having interstitial nephritis and/or interstitial fibrosis, and
wherein the computer is programmed to apply the classifier to a dataset representing a level of expression of marker in a sample of a human individual to thereby classify the test individual into the class representing human subjects having interstitial nephritis fibrosis and/or interstitial fibrosis or the class representing human subjects not having interstitial nephritis and/or interstitial fibrosis, thereby evaluating renal status in a human test subject.

63. A method for profiling gene expression in a human test subject, the method comprising: using a computer, applying a classifier to a sample dataset representing a level of expression in a sample of the test subject of at least two markers selected from the group consisting of: urine monocyte chemotactic protein-1 (uMCP-1), serum creatinine (Src), hepcidin (uHep), and proteinura expressed as a ratio of urine protein:creatine (uPCR), to thereby classify the test subject into a class representing human subjects having interstitial nephritis and/or interstitial fibrosis or a class representing human subjects not having interstitial nephritis and/or interstitial fibrosis,
wherein the classifier is able to discriminate between human subjects having interstitial nephritis and/or interstitial fibrosis and human subjects not having interstitial nephritis and/or interstitial fibrosis,
wherein the classifier is derived from data representing a level of expression of each marker in samples of human subjects having interstitial nephritis and/or interstitial fibrosis and in samples of human subjects not having interstitial nephritis and/or interstitial fibrosis,
and
wherein the computer is programmed to apply the classifier to a dataset representing a level of expression of each marker in a sample of a human individual to thereby classify the human individual into the class representing human subjects having interstitial nephritis and/or interstitial fibrosis or the class representing human subjects not having interstitial nephritis and/or interstitial fibrosis, thereby evaluating renal status in a human test subject.

64. The method of claim 63, further comprising obtaining the sample dataset by measuring the level of expression of each marker in the sample of the test subject, prior to applying the classifier to the sample dataset.

65. The method of claim 60, wherein the classifier is based on a linear Discriminant analysis equation.

66. The method of claim 65, wherein the renal status being evaluated is interstitial nephritis, and
wherein the classifier has a format: Eq(1) Y1=0.992*ln(uMCP1)+2.213*ln(Scr),
where
Y1, if ≧1, classifies the test subject into the class representing human subjects having moderate-severe interstitial nephritis;
Y1, if ≦1, classifies the test subject into the class representing human subjects not having interstitial nephritis or having mild interstitial nephritis;
uMPC1 represents the level of expression of urine monocyte chemoattractant protein-1 (uMPC1) in the sample of the test subject; and
Scr represents the level of serum creatinine (Scr) in the sample of the test subject.

67. The method of claim 65, wherein the renal status being evaluated is interstitial fibrosis, and
wherein the classifier has a format: Eq.2 Y2=4.177*ln(uPCR)−1.425*ln(uHEP),
where
Y2, if ≧−1, classifies the test subject into the class representing human subjects having moderate-severe interstitial fibrosis;
Y2, if ≦−1, classifies the test subject into the class representing human subjects not having interstitial fibrosis or mild interstitial fibrosis;
uHep represents the level of expression of urine hepcidin (uHep) in the sample of the test subject; and
uPCR, proteinura expressed as a ratio of urine protein:creatine (uPCR), represents the level of serum creatinine in the sample of the test subject.

68. The method of claim 4, further comprising obtaining a plurality of classifications for a plurality of samples obtained at a plurality of different times from the subject.

69. The method of claim 13, further comprising obtaining a plurality of classifications for a plurality of samples obtained at a plurality of different times from the subject.

70. The method of claim 17, further comprising obtaining a plurality of classifications for a plurality of samples obtained at a plurality of different times from the subject.

71. A method of claim 31, wherein the method rules in or out an assignment of the subject to early goal-directed therapy.

72. A method of claim 31, wherein the correlating step comprises comparing one or more subject-derived marker concentrations to a predetermined threshold level for a particular marker of interest.

73. A method of claim 31, wherein the correlating step comprises:
determining the concentration of the subject-derived markers,
calculating a single response value based on the concentration of the subject-derived markers, and
comparing the response value to one or more predetermined threshold levels for the response value.

74. A method of claim 31, wherein the correlating step comprises:
comparing the subject-derived marker concentrations to a predetermined threshold level for a particular marker of interest and determining the concentration of the subject-derived markers,
calculating a single response value based on the concentration of each of the subject-derived markers, and
comparing the response value to a predetermined threshold level for the panel response value.

75. A method of claim 13, wherein the sample is from a human.

76. A method of claim 13, wherein the assay method comprises an immunoassay.

77. A method of claim 13, wherein the method rules in or out one or more treatments for inclusion in a therapy regimen comprising administration of immunosuppressive therapy.

78. A method of claim 17, wherein the sample is from a human.

79. A method of claim 17, wherein the assay method comprises an immunoassay.

80. A method of claim 17, wherein the method rules in or out one or more treatments for inclusion in a therapy regimen comprising administration of immunosuppressive therapy.

81. A method of claim 28, wherein the sample is from a human.

82. A method of claim 28, wherein the assay method comprises an immunoassay.

83. A method of claim 28, wherein the method rules in or out one or more treatments for inclusion in a therapy regimen comprising administration of immunosuppressive therapy.

84. A method of claim 29, wherein the sample is from a human.

85. A method of claim 29, wherein the assay method comprises an immunoassay.

86. A method of claim 29, wherein the method rules in or out one or more treatments for inclusion in a therapy regimen comprising administration of immunosuppressive therapy.

87. A method of claim 31, wherein the sample is from a human.

88. A method of claim 31, wherein the assay method comprises an immunoassay.

89. A method of claim 31, wherein the method rules in or out one or more treatments for inclusion in a therapy regimen comprising administration of immunosuppressive therapy.

90. The method of claim 62, wherein the classifier is based on a linear Discriminant analysis equation.

91. The method of claim 63, wherein the classifier is based on a linear Discriminant analysis equation.
